(19) United States
(12) Patent Application Publication
(10) Pub. No.: US 2010/0247548 A1
(43) Pub. Date: Sep. 30, 2010

(54) ANTAGONISTS OF CI-M6P/IGF2R FOR PREVENTION AND TREATMENT OF CTGF-MEDIATED OCULAR DISORDERS

(75) Inventors: Allan R. Shepard, Fort Worth, TX (US); Debra L. Fleenor, Crowley, TX (US); Abbot F. Clark, Arlington, TX (US); Najam A. Sharif, Fort Worth, TX (US)

Correspondence Address: ALCON, IP LEGAL, TB4-8, 6201 SOUTH FREEWAY, FORT WORTH, TX, 76134, US

(73) Assignee: Alcon Research, Ltd., Fort Worth, TX (US)

(21) Appl. No.: 12/796,748

(22) Filed: Jun. 9, 2010

Related U.S. Application Data
(63) Continuation of application No. 11/847,523, filed on Aug. 30, 2007.
(60) Provisional application No. 60/841,405, filed on Aug. 31, 2006.

(51) Int. Cl. A61K 39/395; A61K 38/00; A61K 31/70; A61K 31/7088; A61K 31/175; A61P 27/02

(52) U.S. Cl. 424/158.1; 514/20.8; 514/25; 514/44 R; 514/592

(57) ABSTRACT

Antagonists of cation-independent mannose 6-phosphate/insulin-like growth factor-II receptor are provided for attenuation of CTGF signaling in a method of down-regulation of receptor signaling and downstream decreased signaling of connective tissue growth factor in ocular disorders involving inappropriate CTGF signaling. Ocular disorders involving inappropriate CTGF signaling include ocular hypertension, glaucoma, glaucomatous retinopathy, optic neuropathy, macular degeneration, diabetic retinopathy, choroidal neovascularization, and proliferative vitreoretinopathy, for example. Such disorders are treated by administering antagonists of the present invention.

### CROSS-REFERENCE TO RELATED APPLICATION

This application claims priority under 35 U.S.C. §119 to U.S. Provisional Patent Application No. 60/841,405 filed Aug. 31, 2006, the entire contents of which are incorporated herein by reference.

### FIELD OF THE INVENTION

The present invention relates to the field of compositions for attenuation of cation-independent mannose 6-phosphate/insulin-like growth factor-II receptor (CI -M6P/IGF2R) for down-regulation of receptor signaling and downstream decreased effects of connective tissue growth factor (CTGF) action in ocular disorders involving CTGF.

### BACKGROUND OF THE INVENTION

Most ocular disorders are associated with cellular processes including cell proliferation, survival, migration, differentiation, and angiogenesis. CTGF is a secreted cytokine believed to be a central mediator in these cellular processes. In particular, CTGF is known to increase extracellular matrix production via increased deposition of collagen and fibronectin. Overexpression of CTGF has been implicated as a major causative factor in conditions such as scleroderma, fibroproliferative diseases, and scarring in which there is an over accumulation of extracellular matrix components.

An over accumulation of extracellular matrix materials in the region of the trabecular meshwork (TM) is a hallmark of certain forms of glaucoma; such increase's are believed to lead to increased resistance to aqueous outflow and, therefore, elevated intraocular pressure (IOP). International Patent Application No. PCT/US2003/012521 to Fleenor et al. published Nov. 13, 2003 as WO 03/092584 and assigned to Alcon, Inc. describes the elevated presence of CTGF mRNA in glaucomatous TM cells vs. normal TM cells. Thus, it is believed that CTGF plays a role in extracellular matrix production by the trabecular meshwork cells.

The TM is a complex tissue including trabecular cells, connective tissue, and extracellular matrix located at the angle between the cornea and iris that provides the normal resistance required to maintain a normal IOP. An adequate IOP is needed to maintain the shape of the eye and to provide a pressure gradient to allow for the flow of aqueous humor to the avascular cornea and lens. Excessive IOP, commonly present in glaucoma, has deleterious effects on the optic nerve, leads to loss of retinal ganglion cells and axons, and results in progressive visual loss and blindness if not treated. Glaucoma is one of the leading causes of irreversible visual impairment and blindness worldwide.

Most forms of glaucoma result from disturbances in the flow of aqueous humor that have an anatomical, biochemical or physiological basis. Primary open angle glaucoma (POAG), also known as chronic or simple glaucoma, represents the majority of all glaucomas in the United States. POAG is characterized by pathological changes in the TM, resulting in abnormally high resistance to fluid drainage from the eye. A consequence of such resistance is an increase in the IOP.

Certain drugs such as prednisone, dexamethasone, and hydrocortisone are known to induce glaucoma in some individuals by increasing IOP. Further, the mode of administration appears to affect IOP. For example, ophthalmic administration of dexamethasone leads to greater increases in IOP than does systemic administration. Glaucoma that results from the administration of steroids is termed steroid-induced glaucoma.

Current anti-glaucoma therapies lower IOP by the use of medications to suppress aqueous humor formation or to enhance aqueous outflow, as well as surgical procedures, such as laser trabeculoplasty, or trabeculectomy, to improve aqueous drainage. Pharmaceutical anti-glaucoma approaches have exhibited various undesirable side effects. For example, miotics such as pilocarpine can cause blurring of vision and other negative local, side effects. Systemically administered carbonic anhydrase inhibitors can cause nausea, dyspepsia, fatigue, and metabolic acidosis. Further, certain beta-blockers have been associated with pulmonary side effects attributable to their effects on beta-2 receptors in pulmonary tissue. Alpha-2-agonists can cause tachycardia, arrhythmia and hypertension. Such negative side effects may lead to decreased patient compliance or to termination of therapy.

U.S. Published Patent Application No. 2005/0234075 to Fleenor et al., published Oct. 20, 2005, hereby incorporated by reference herein, provides GSK-3 and CDK inhibitors having inhibitory activity for both basal and TGFβ2-induced CTGF expression in human trabecular meshwork cells.

Macular degeneration (AMD) is the loss of photoreceptors in the portion of the central retina, termed the macula, responsible for high-acuity vision. Degeneration of the macula is associated with abnormal deposition of extracellular matrix components and other debris in the membrane between the retinal pigment epithelium and the vascular choroid. This debris-like material is termed drusen. Drusen is observed with a funduscopic eye examination. Normal eyes may have maculas free of drusen, yet drusen may be abundant in the retinal periphery. The presence of soft drusen in the macula, in the absence of any loss of macular vision, is considered an early stage of AMD.

Choroidal neovascularization (CNV) commonly occurs in macular degeneration in addition to other ocular disorders and is associated with proliferation of choroidal endothelial cells, overproduction of extracellular matrix, and formation of a fibrovascular subretinal membrane. Retinal pigment epithelium cell proliferation and production of angiogenic factors appears to effect choroidal neovascularization.

Diabetic retinopathy (DR) is an ocular disorder that develops in diabetes due to thickening of capillary basement membranes and lack of contact between pericytes and endothelial cells of the capillaries. Loss of pericytes increases leakage of the capillaries and leads to breakdown of the blood-retina barrier.

Proliferative vitreoretinopathy is associated with cellular proliferation of cellular and fibrotic membranes within the vitreous membranes and on the surfaces of the retina. Retinal pigment epithelium cell proliferation and migration is common with this ocular disorder. The membranes associated with proliferative vitreoretinopathy contain extracellular matrix components such as collagen types I, II, and IV and fibronectin, and become progressively fibrotic.

In view of the importance of the above-cited ocular disorders, particularly the pathological damage due to overproduction of extracellular matrix, it is desirable to have an improved method of treating these ocular disorders that addresses underlying causes of its progression.

### Abbreviations as Used Herein Include

CI Cation independent
CI-M6P/IGF2 Cation independent mannose 6-phosphate/insulin growth factor-2
CI-M6P/IGF2R Cation independent mannose 6-phosphate/insulin growth factor-2 receptor
CTGF Connective tissue growth factor
IGF2 or IGFII Insulin growth factor-2
IGF2R or IGFIIR Insulin growth factor-2 receptor
IOP Intraocular pressure
M6P Mannose 6-phosphate
TGFβ Transforming growth factor β
TGFβR Transforming growth factor β receptor
UPA Urokinase-type plasminogen activator.

### SUMMARY OF THE INVENTION

The present invention addresses the above-cited problems in the art and provides a method for attenuating CTGF signaling in an eye of a subject by providing antagonists of the CI-M6P/IGF2 receptor. A method of attenuating CTGF signaling in an eye of a subject comprises administering to the subject a composition comprising an effective amount of an antagonist of the CI-M6P/IGF2 receptor or a pharmaceutically acceptable salt or prodrug thereof, and a pharmaceutically acceptable carrier. CTGF signaling in the eye of the subject is attenuated thereby. The subject may have a CTGF signaling-associated ocular disorder resulting in inappropriate connective tissue growth factor signaling or may be at risk of developing such an ocular disorder. The CTGF signaling-associated ocular disorder may be ocular hypertension, glaucoma, glaucomatous retinopathy, optic neuropathy, macular degeneration, diabetic retinopathy, choroidal neovascularization, or proliferative vitreoretinopathy, for example.

The antagonist of CI-M6P/IGF2 receptor decreases signaling by the receptor. The antagonist may comprise a mannose-6-phosphate analog, fructose-1-phosphate, a fructose-1-phosphate analog, a polysulfonated naphthylurea such as suramin; or a polynucleotide, peptidomimetic, peptide, antibody, or biologically active fragment thereof having binding specificity and affinity for latent TGFβ2, CTGF, IGFII, or CI -M6P/IGF2R.

Another embodiment of the invention is a method of treating a CTGF signaling-associated ocular disorder associated with inappropriate connective tissue growth factor signaling in a subject in need thereof. The method comprises administering to the subject a composition comprising an effective amount of an antagonist of CI -M6P/IGF2 receptor or a pharmaceutically acceptable salt or prodrug thereof, and a pharmaceutically acceptable carrier. The CTGF signaling-associated ocular disorder is treated thereby.

In one embodiment of the invention, a method of treating glaucoma in a subject is provided. The method comprises administering to the subject a composition comprising an effective amount of an antagonist of CI-M6P/IGF2 receptor or a pharmaceutically acceptable salt or prodrug thereof, and a pharmaceutically acceptable carrier, wherein the glaucoma is treated thereby.

In another embodiment of the present invention a method of treating glaucomatous retinopathy, optic neuropathy, macular degeneration, diabetic retinopathy, choroidal neovascularization, or proliferative vitreoretinopathy in a subject is provided. The method comprises administering to the subject a composition comprising an effective amount of an antagonist of CI-M6P/IGF2 receptor or a pharmaceutically acceptable salt or prodrug thereof, and a pharmaceutically acceptable carrier. The glaucomatous retinopathy, optic neuropathy, macular degeneration, diabetic retinopathy, choroidal neovascularization, or proliferative vitreoretinopathy is treated thereby.

### DETAILED DESCRIPTION OF THE INVENTION

Mammalian cells possess two types of M6P receptors: the cation independent (CI) mannose 6-phosphate receptor, also known as the insulin-like growth factor receptor II (IGF-IIR) and the cation-dependent mannose 6-phosphate receptor. Embodiments of the present invention relate to antagonizing CTGF signaling activity mediated via the cation-independent mannose 6-phosphate/insulin-like growth factor-II receptor for the prevention and treatment of CTGF-related ocular disorders.

CI-M6P/IGF2R is an oligomeric. ≈250-300-kDa multifunctional transmembrane glycoprotein having binding sites for a variety of ligands including mannose-6-phosphate, IGF2, urokinase-type plasminogen activator (uPA) receptor, plasminogen, latent TGFβ, retinoic acid, and granzyme B. The receptor has a signal sequence, an extra-cytoplasmic domain including 15 conserved regions, a transmembrane region, and a cytoplasmic domain. The receptors are primarily present intracellularly and the rest are present at the cell surface. The extracellular receptors bind extracellular ligands, such as IGF2 thereby mediating endocytosis of IGF2, for example. The intracellular receptors are involved in the sorting and transporting of M6P-bearing glycoproteins from the trans-Golgi network to endosomes. In the absence of M6P receptors, M6P-containing glycoproteins are generally secreted from the cell. The CI -M6P/IGF2R also participates in activation of latent transforming growth factor possibly via uptake of uPA, which may mediate conversion of plasminogen to plasmin, resulting in the activation of TGFβ. Further contributing to the multifunctional nature of the CI -M6P/IGF2R is the reported identification of the CTGF receptor in corneal fibroblasts as the type II IGF receptor (T. Blalock, Ph.D. thesis, Univ. of Florida, 8/03).

TGFβ is known to increase the expression of CTGF (Xin et al., JBC, Vol. 279(34):35255-35262, 2004; Katsuma et al., FEBS Letters, Vol. 579:2576-2582, 2005), a protein that appears to be a key player in the glaucoma process (International Patent Application No. PCT/US2003/012521 to Fleenor et al. published Nov. 13, 2003 as WO 03/092584 and assigned to Alcon, Inc.). Significantly higher levels of TG932 isoform has been found in aqueous humor collected from glaucomatous human eyes as compared to “normal” eyes (Tripathi et al., Exp Eye Res, Vol. 59(6):723-727, 1994; Inatani et al., Graefes Arch Clin Exp Opthalmol, Vol. 239(2):109-113, 2001; Picht et al., Graefes Arch Clin Exp Opthalmol, Vol. 239(3):199-207, 2001; Ochiai et al., Japan J Opthalmol, Vol. 46(3):249-253, 2002). Furthermore, TGFβ2 is able to provoke substantial increases in IOP in a perfused human anterior segment model (Fleenor et al., Invest Opthalmol V is Sci, Vol. 47(1):226-234, 2006). Therefore, TGFβ, in particular TGFβ2, appears to have a causative role in IOP-related disorders such as glaucoma.

The present inventors provide herein methods for targeting the downstream effects of CTGF action in ocular disorders such as glaucoma by interfering with the binding of CTGF to the CI-M6P/IGF2R or interfering with the subsequent signaling of the complex. While not wanting to be bound by theory, a feedback scheme for signaling is provided as follows.

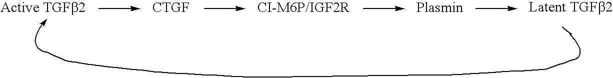

CTGF may interact with CI-M6P/IGF2R either on the cell surface or intracellularly in the ER-Golgi. Inhibition of CTGF binding and/or signaling via the CI-M6P/IGF2R is provided herein as decreasing levels of active TGFβ, thereby interfering with the positive feedback in the scheme provided supra, and is useful in ocular disorders having inappropriate CTGF signaling such as in glaucoma, CNV, AMD, and DR, particularly proliferative DR. Inhibition of IGF2 binding and/or signaling via the CI-M6P/IGF2R is also provided since IGF2 binds to the receptor, albeit to a different domain.

Antagonists of cation-independent mannose 6-phosphate/insulin-like growth factor-II receptor (CI-M6P/IGFII-R): Antagonists of the cation-independent mannose 6-phosphate/insulin-like growth factor-II receptor include agents that attenuate binding affinity or specificity between the receptor and its binding ligands, CTGF, IGF-2, or latent TGFβ2. Antagonists include a mannose 6-phosphate analog, fructose-1-phosphate, a fructose-1-phosphate analog, a polysulfonated naphthylurea such as suramin (most commonly available as the hexasodium salt), a polynucleotide, peptide, peptidomimetic, antibody, or biologically active fragment thereof having binding specificity and affinity for the CI-M6P/IGFII receptor or one of its binding ligands, CTGF, IGF-2, or latent TGFβ2; or a pharmaceutically acceptable salt or prodrug of an antagonist. Antagonists may cause an inhibition of the constitutive activity of the receptor; such drugs are not technically antagonists but are agonists with a negative intrinsic activity. These drugs are called inverse agonists and are included in the term “antagonist,” as used herein. That is, an antagonist may be an agent that stabilizes an inactive form of the CI-M6P/IGF2R and thereby prevents signaling of the basal or the ligand-bound receptor.

As used herein, a “pharmaceutically acceptable salt” refers to a salt of an antagonist that retains the function of the CI-M6P/IGFII receptor antagonist and that is compatible with administration as desired. A salt may be formed from an acid or a base depending upon the nature of the antagonist. A salt may be formed with an acid such as acetic acid, benzoic acid, cinnamic acid, citric acid, ethanesulfonic acid, fumaric acid, glycolic acid, hydrobromic acid, hydrochloric acid, maleic acid, malonic acid, mandelic acid, methanesulfonic acid, nitric acid, oxalic acid, phosphoric acid, propionic acid, pyruvic acid, salicylic acid, succinic acid, sulfuric acid, tartaric acid, p-toluenesulfonic acid, trifluoroacetic acid, and the like. A salt may be formed with a base such as a primary, secondary, or tertiary amine, aluminum, ammonium, calcium, copper, iron, lithium, magnesium, manganese, potassium, sodium, zinc, and the like.

As used herein, the term “prodrug” refers to a derivative of an antagonist that has minimal therapeutic activity until it is converted to its desired biologically active form. A prodrug is an antagonist having one or more functional groups or carriers covalently bound thereto, which functional groups or carriers are removed from the compound by metabolic processes within the body to form the respective bioactive antagonist. Prodrugs of antagonists of the present invention are prepared by modifying functional groups present in the antagonists in such a way that the modifications are hydrolyzed, oxidized, or otherwise reacted, either in routine manipulation or in vivo, to yield the desired antagonist. Prodrugs include alcohols, amides, amines, carbamates, carbonates, esters, nitrites, nitrates, nitroso, sulfates, sulfites, sulfhydryl, ureides, and phosphate derivatives, for example.

In an embodiment of the invention, the antagonist is a mannose-6-phosphate analog having structure I:

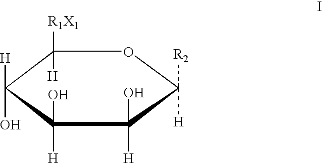

wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀)alkylamido, (C₁-C₂₀)alkylamino, or (C₁-C₂₀)alkylcarboxy.
   The dotted lines of structure I indicate that R₂ is axial or equatorial.

In one embodiment of the invention, the antagonist has structure I where R₁ is C₁-C₂ alkyl, X₁ is phosphonate or carboxy, and R₂ is hydroxy or methoxy. In another embodiment of the invention R₁ is C₂ haloalkyl, C₁ hydroxyalkyl, C₂ alkenyl or C₂ haloalkenyl; X₁ is phosphonate; and R₂ is hydroxyl or methoxy.

In another embodiment of the invention, the antagonist is fructose 1-phosphate, or a fructose-1-phosphate analog having structure II:

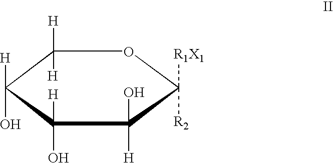

wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀) alkylamido, (C₁-C₂₀)alkylamino, or (C₁-C₂₀)alkylcarboxy.
   The dotted lines of structure II indicate that R₁X₁ and R₂ may be axial or equatorial. One of ordinary skill in the art would realize that fructose derivatives may adopt a 5-membered ring configuration in addition to the 6-membered ring configuration shown above.

As used herein “phosphate analog” includes the terms phosphorothioate, dithioate, -selenoate, -disclenoate, -anilothioate, -anilidate, -amidate, or boron phosphate, for example. Representative examples of alkyl, alkenyl, and alkynyl groups include straight-chain, branched or cyclic isomers. A substituted alkyl has one or more functional groups as substituents. Among the halo substituents, fluoro, chloro, and bromo are particularly contemplated herein. The term “hydroxyalkyl” is meant to include alcohols, glycols and diols of alkyls. Representative examples of alkoxy groups include the alkyl groups as herein described having ether linkages.

An assay for identifying further antagonists of CI-M6P/IGF2 receptor uses a competitive binding assay which may comprise combining a candidate antagonist, labeled CTGF or IGF-2, CI-M6P/IGF2 receptor and measuring the amount of labeled material associated with the receptor. The result is compared with the amount of labeled material associated with the receptor using the same assay in the absence of the candidate antagonist. The candidate antagonist has antagonist activity when the level of labeled material associated with the receptor is lower than when the candidate is not present. Further assays may include assays for inhibition of receptor specific antibody binding by a candidate antagonist, reduced accumulation of a CTGF-induced mRNA by a candidate antagonist, or reduced accumulation of a CTGF-induced protein by a candidate antagonist.

Phosphonate analogues are synthesized using methods known in the art, for example, methods described by Ferguson et al. (U.S. Pat. No. 6,140,307 issued Oct. 31, 2000, which patent is incorporated by reference herein). Methods of synthesis for difluorovinylphosphonates, related monofluorophosphonates, and hydroxyphosphonates are described by Berkowitz, J. Org. Chem., Vol. 65:4498, 2000. Methods of synthesis of gluco epimers of fluorovinylphosphonates are described by Gross, Tetrahedron Letters, Vol. 34:7197, 1993. Phosphate analogs, sulfates, and sulfonates are synthesized in a similar manner using the appropriate reactants as is readily determined by one of ordinary skill in the art of organic synthesis. Sulfate and carboxylate analogues are synthesized using, for example, methods as set forth by Vidal et al., Bioorganic & Medicinal Chemistry, Vol. 10:4051, 2002, Clavel et al., Il Farmaco, Vol. 60:721-725, 2005, and Jeanjean et al., Bioorganic & Medicinal Chemistry, Vol. 14:3375, 2006. Vidil, Eur. J. Org. Chem, Vol. 2:477, 1999 details the synthesis of O-methyl glycosides. Further analogues are synthesized as described in U.S. patent application 2003/0176363 published Sep. 18, 2003 (U.S. Ser. No. 10/338,679 filed Jan. 9, 2003) and International PCT application published as WO 2004/104015, Dec. 2, 2004 (PCT/US2004/015876 to Cowden et al.) which applications are incorporated by reference herein in their entirety.

Antibodies having binding specificity and affinity for the CI-M6P/IGF2 receptor are available commercially, for example, catalog no. ab2733 that recognizes an epitope in the extracellular domain of the receptor (mouse monoclonal 2G11), catalog no. ab12894 (rabbit polyclonal), and catalog no. ab32815 (rabbit polyclonal); all from to ABCAM® (#ab13210, Cambridge, Mass.).

Peptides having antagonistic activity include a synthetic peptide derived from residues 700-800 of the human CI-M6P/IGF2R that competitively binds CTGF, for example, available from ABCAM® (#ab13210, Cambridge, Mass.).

Antagonism of CI-M6P/IGF2 receptors and resultant inhibition of CTGF signaling is also inferred in a human or mammal by observing an improvement in an ocular disorder. For example, in age-related macular degeneration a slowing or reversal of vision loss indicates inhibition of CTGF signaling and, in glaucoma patients, lowered intraocular pressure and a delay or prevention of the onset of symptoms in a subject at risk for developing glaucoma indicates inhibition of CTGF signaling.

Antagonists of the present invention may be used in combination with other agents for treating ocular disorders where CTGF accumulation or activity is inappropriate such as, for example, agents described by U.S. Published Patent Application No. 2005/0234075 to Fleenor et al., published Oct. 20, 2005, previously incorporated by reference herein.

Mode of administration: The antagonist may be delivered directly to the eye (for example: topical ocular drops or ointments; slow release devices in the cul-de-sac or implanted adjacent to the sclera (transscleral) or within the eye; periocular, conjunctival, sub-Tenons, intracameral, intravitreal, sub-retinal, retrobulbar, or intracanalicular injections) or systemically (for example: oral; intravenous, subcutaneous or intramuscular injections; parenterally, dermal delivery) using techniques well known by those skilled in the art. It is further contemplated that the antagonists of the invention may be formulated in a placement device such as a retinal pellet, intraocular insert, catheter, suppository or an implant device comprising a porous, non-porous, or gelatinous material. Intracameral injection may be through the cornea into the anterior chamber to allow the agent to reach the trabecular meshwork. Intracanalicular injection may be into the venous collector channels draining Schlemm's canal or into Schlemm's canal.

Subject: A subject in need of treatment for an ocular disorder or at risk for developing an ocular disorder is a human or other mammal having a condition or at risk of having a condition associated with inappropriate signaling by CTGF. Such an ocular disorder may include, for example, ocular hypertension, glaucoma, macular degeneration, diabetic retinopathy, choroidal neovascularization, proliferative vitreoretinopathy, and conditions with endothelial cell proliferation, or fibroproliferation. Ocular structures associated with such disorders may include the retina, choroid, lens, trabecular meshwork, rod, cone, RPE, ganglia, macula, iris, sclera, aqueous chamber, vitreous chamber, ciliary body, optic disc, optic nerve, papilla, or fovea, for example.

Formulations and Dosage: Pharmaceutical formulations comprise an antagonist, or salt thereof, as set forth herein up to 99% by weight mixed with a physiologically acceptable ophthalmic carrier medium such as water, buffer, saline, glycine, hyaluronic acid, mannitol, and the like. Examples of possible formulations embodied by aspects of the invention are as follows.

| Compound | Amount in weight % |
|---|---|
| CI-M6P/IGF2 receptor antagonist | up to 99; 0.1-99; 0.1-50; 0.5-10.0; |
| | 0.01-5.0; 0.01-2.0; 0.02-2.0; 0.1-1.0; |
| | 0.5-2.0 |
| Hydroxypropylmethylcellulose | 0.5 |
| Sodium chloride | .8 |
| Benzalkonium Chloride | 0.01% |
| EDTA | 0.01 |
| NaOH/HCl | qs pH 7.4 |
| Purified water | qs 100 mL |

| Compound | Amount in weight % |
|---|---|
| CI-M6P/IGF2 receptor antagonist | up to 99; 0.1-99; 0.1-50; 0.5-10.0; |
| | 0.01-5.0; 0.01-2.0; 0.02-2.0; 0.1-1.0; |
| | 0.5-2.0; 0.00005-0.5; 0.0003-0.3; |
| | 0.0005-0.03; 0.001 |
| Phosphate Buffered Saline | 1.0 |
| Benzalkonium Chloride | 0.01 |
| Polysorbate 80 | 0.5 |
| Purified water | q.s. to 100% |

| Compounds | Amount in weight % |
|---|---|
| CI-M6P/IGF2 receptor antagonist | up to 99; 0.1-99; 0.1-50; 0.5-10.0; |
| | 0.01-5.0; 0.01-2.0; 0.02-2.0; 0.1-1.0; |
| | 0.5-2.0; 0.001 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water | q.s. to 100% |

| Compounds | Amount in weight % |
|---|---|
| CI-M6P/IGF2 receptor antagonist | up to 99; 0.1-99; 0.1-50; 0.5-10.0; |
| | 0.01-5.0; 0.01-2.0; 0.02-2.0; 0.1-1.0; |
| | 0.5-2.0; 0.0005 |
| Phosphate Buffered Saline | 1.0 |
| Hydroxypropyl-β-cyclodextrin | 4.0 |
| Purified water | q.s. to 100% |

In a further embodiment, the ophthalmic compositions are formulated to provide for an intraocular concentration of about 0.1-100 micromolar (μM) or, in a further embodiment, 1-100 nM of the antagonist. Topical compositions are delivered to the surface of the eye one to four times per day according to the routine discretion of a skilled clinician. The pH of the formulation should be pH 4-pH 9, or about pH 4.5 to about pH 7.4. Systemic formulations may contain about 10 to 1000 mg of the antagonist.

An “effective amount” refers to that amount of CI-M6P/IGF2 receptor antagonist that is able to disrupt binding and/or subsequent signaling between the CI -M6P/IGF2 receptor and CTGF via the feedback loop cited supra. Such disruption leads to lowered CTGF signaling activity, and resultant lessening of symptoms in ocular disorders in a subject. Such disruption delays or prevents the onset of symptoms in a subject at risk for developing ocular disorders as set forth herein. The effective amount of a formulation may depend on factors such as the age, race, and sex of the subject, or the severity of the ocular condition, for example. In one embodiment, the antagonist is delivered topically to the eye and reaches the trabecular meshwork, retina or optic nerve head at a therapeutic dose thereby ameliorating the ocular disease process.

Acceptable carriers: An ophthalmically acceptable carrier refers to those carriers that cause at most, little to no ocular irritation, provide suitable preservation if needed, and deliver one or more CI-M6P/IGF2R antagonists of the present invention in a homogenous dosage. For ophthalmic delivery, a CI-M6P/IGF2R antagonist may be combined with opthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the antagonist in a physiologically acceptable isotonic aqueous buffer. Further, the ophthalmic solution may include an opthalmologically acceptable surfactant to assist in dissolving the antagonist. Viscosity building agents, such as hydroxymethyl cellulose, hydroxyethyl cellulose, methylcellulose, polyvinylpyrrolidone, or the like, may be added to the compositions of the present invention to improve the retention of the compound.

In order to prepare a sterile ophthalmic ointment formulation, the CI -M6P/IGF2R antagonist is combined with a preservative in an appropriate vehicle, such as mineral oil, liquid lanolin, or white petrolatum. Sterile ophthalmic gel formulations may be prepared by suspending the CI-M6P/IGF2R antagonist in a hydrophilic base prepared from the combination of, for example, CARBOPOL®-940 (BF Goodrich, Charlotte, N.C.), or the like, according to methods known in the art for other ophthalmic formulations. VISCOAT® (Alcon Laboratories, Inc., Fort Worth, Tex.) may be used for intraocular injection, for example. Other compositions of the present invention may contain penetration enhancing agents such as cremophor and TWEEN® 80 (polyoxyethylene sorbitan monolaureate, Sigma Aldrich, St. Louis, Mo.), in the event the CI-M6P/IGF2R antagonists are less penetrating in the eye.

Kits: Embodiments of the present invention provide a kit that includes to antagonists for attenuating CTGF-mediated CI-M6P/IGF2R receptor signaling in a cell. The kit contains in close confinement one or more containers containing an antagonist of the present invention, a pharmaceutically acceptable carrier and, optionally, printed instructions for use.

### Example 1

### Inhibition of CI-M6P/IGF2R-Mediated Signaling

The effect of CI-M6P/IGF2 receptor antagonism on expression of extracellular matrix-related proteins by cultured human trabecular meshwork cells is determined as follows. Human TM cell cultures are split into replicate and/or experimental and/or control groups to which are then added control solutions or experimental solutions comprising diluent vehicle(s) (as controls) and/or CTGF (as stimulatory agent) and/or CI-M6P/IGF2 receptor antagonists. Levels of extracellular matrix-related proteins, such as fibronectin, plasminogen activator inhibitor I (PAI-1), collagens, fibrillin, vitronectin, laminin, thrombospondin I, proteoglycans, or integrins, are then measured in each cell culture group via standard enzyme-linked immunoabsorbent assays (ELISA). Such assays are well-known to those skilled in the art and are sensitive immunoassays which utilize an enzyme linked to an antibody or antigen as a marker for the detection of a specific protein. By these means, levels of various extracellular matrix-related proteins can then be compared between the groups in order to determine the effect of CI-M6P/IGF2R antagonists.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more.”

What is claimed is:

1. A method of attenuating CTGF signaling in an eye of a subject, comprising:
administering to the subject a composition comprising:
an effective amount of an antagonist of CI-M6P/IGF2R, or a pharmaceutically acceptable salt or prodrug thereof; and
a pharmaceutically acceptable carrier;
to wherein CTGF signaling in the eye of the subject is attenuated thereby.

2. The method of claim 1 wherein the subject has a CTGF signaling-associated ocular disorder with inappropriate connective tissue growth factor activity.

3. The method of claim 1 wherein the subject is at risk of developing a CTGF signaling-associated ocular disorder with inappropriate connective tissue growth factor activity.

4. The method of claim 2 wherein the CTGF signaling-associated ocular disorder is ocular hypertension, glaucoma, glaucomatous retinopathy, optic neuropathy, macular degeneration, diabetic retinopathy, choroidal neovascularization, or proliferative vitreoretinopathy.

5. The method of claim 1 wherein the antagonist is a mannose-6-phosphate analog, fructose-1-phosphate, a fructose-1-phosphate analog, a polysulfonated naphthylurea; or a polynucleotide, peptidomimetic, peptide, antibody, or biologically active fragment thereof having binding specificity and affinity for CTGF, IGFII, latent TGFβ2 or CI-M6P/IGF2R.

6. The method of claim 1 wherein the antagonist is a mannose-6-phosphate analog having structure I: 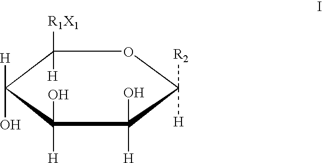
wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀)alkylamido, (C₁-C₂₀)alkylamino, or (C₁-C₂₀)alkylcarboxy; and wherein R₂ is axial or equatorial.

7. The method of claim 6 wherein R₁ is C₁-C₂ alkyl and X₁ is phosphonate.

8. The method of claim 6 wherein R₁ is C₂ haloalkyl and X₁ is phosphonate.

9. The method of claim 6 wherein R₁ is C₁ hydroxyalkyl and X₁ is phosphonate.

10. The method of claim 6 wherein R₁ is C₂ alkenyl or C₂ haloalkenyl and X₁ is phosphonate.

11. The method of claim 1 wherein the antagonist is fructose 1-phosphate or an analog thereof having structure II: 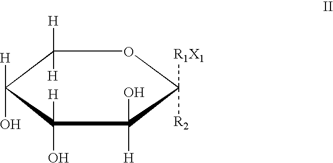
wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀)alkylamido, (C₁-C₂₀)alkylamino, or (C₁-C₂₀)alkylcarboxy; and wherein R₂ is axial or equatorial.

12. The method of claim 1 wherein the antagonist is a polysulfonated naphthylurea.

13. The method of claim 1 wherein the antagonist is a polynucleotide or a biologically active fragment thereof having binding affinity and specificity for CI -M6P/IGF2R.

14. The method of claim 1 wherein the antagonist is an antibody or a biologically active fragment thereof having binding affinity and specificity for CI-M6P/IGF2R.

15. The method of claim 1 wherein the antagonist is a peptide or peptidomimetic having binding affinity and specificity for CI-M6P/IGF2R.

16. The method of claim 1 wherein the composition is administered via a topical, intracameral, intravitreal, transcleral, or an implant route.

17. The method of claim 1 wherein the concentration of the antagonist in the composition is from 0.01% to 2%.

18. A method of treating a CTGF signaling-associated ocular disorder in a subject in need thereof, comprising:
administering to the subject a composition comprising:
an effective amount of an antagonist of CI-M6P/IGF2R, or a pharmaceutically acceptable salt or prodrug thereof; and
a pharmaceutically acceptable carrier;
wherein the CTGF signaling-associated ocular disorder is treated thereby.

19. The method of claim 18 wherein the subject has ocular hypertension or to glaucoma.

20. The method of claim 18 wherein the subject is at risk of developing ocular hypertension or glaucoma.

21. The method of claim 18 wherein the antagonist is a mannose-6-phosphate analog, fructose-1-phosphate, a fructose-1-phosphate analog, a polysulfonated naphthylurea; or a polynucleotide, peptidomimetic, peptide, antibody, or biologically active fragment thereof having binding specificity and affinity for CTGF, IGFII, latent TGFβ2 or CI-M6P/IGF2R.

22. The method of claim 18 wherein the antagonist is a mannose-6-phosphate analog having structure I: 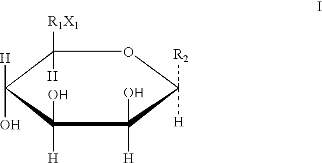
wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀)alkylamido, (C₁-C₂₀)alkylamino, or (C₁-C₂₀)alkylcarboxy; and wherein R₂ is axial or equatorial.

23. The method of claim 22 wherein R₁ is C₁-C₂ alkyl and X₁ is phosphonate.

24. The method of claim 22 wherein R₁ is C₂ haloalkyl and X₁ is phosphonate.

25. The method of claim 22 wherein R₁ is C₁ hydroxyalkyl and X₁ is phosphonate.

26. The method of claim 22 wherein R₁ is C₂ alkenyl or C₂ haloalkenyl and X₁ is phosphonate.

27. The method of claim 18 wherein the antagonist is fructose 1-phosphate, or a fructose-1-phosphate analog having structure II: 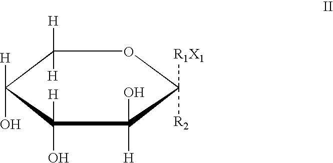
wherein
R₁ is C₁-C₃ alkyl, C₁-C₃ hydroxyalkyl, C₁-C₃ haloalkyl, C₂-C₃ alkenyl, C₂-C₃ alkoxy or C₂-C₃ haloalkenyl;
X₁ is phosphonate, phosphate analog, sulfate, sulfonate, carboxy, di-carboxy or monoester thereof; and
R₂ is hydroxy, cyano; or optionally substituted C₂-C₂₀ alkyl, C₂-C₂₀ alkenyl, C₂-C₂₀ alkynyl, C₂-C₂₀ alkoxy, aryl, heteroaryl, aryl(C₁-C₂₀)alkyl, heteroaryl(C₁-C₂₀)alkyl, (C₁-C₂₀)oxyalkyl, (C₁-C₂₀)alkylamido, (C₁-C₂₀)alkylamino, or (C₁-C₂₀)alkylcarboxy; and wherein R₂ is axial or equatorial.

28. The method of claim 18 wherein the antagonist is a polysulfonated naphthylurea.

29. The method of claim 18 wherein the antagonist is a polynucleotide or a biologically active fragment thereof having binding affinity and specificity for CI -M6P/IGF2R.

30. The method of claim 18 wherein the antagonist is an antibody or a biologically active fragment thereof having binding affinity and specificity for CI-M6P/IGF2R.

31. The method of claim 18 wherein the antagonist is a peptide or peptidomimetic having binding affinity and specificity for CI-M6P/IGF2R.

32. The method of claim 18 wherein the composition is administered via a topical, intracameral, intravitreal, transcleral, or an implant route.

33. The method of claim 18 wherein the concentration of the antagonist in the composition is from 0.01% to 2%.

34. A method of treating glaucoma in a subject, comprising:
administering to the subject a composition comprising:
an effective amount of an antagonist of CI-M6P/IGF2R, or a pharmaceutically acceptable salt or prodrug thereof; and
a pharmaceutically acceptable carrier;
wherein the glaucoma is treated thereby.

35. A method of treating glaucomatous retinopathy, optic neuropathy, macular degeneration, diabetic retinopathy, choroidal neovascularization, or proliferative vitreoretinopathy in a subject, comprising:
administering to the subject a composition comprising:
an effective amount of an antagonist of CI-M6P/IGF2R or a pharmaceutically acceptable salt or prodrug thereof; and
a pharmaceutically acceptable carrier;
wherein the glaucomatous retinopathy, optic neuropathy, macular degeneration, diabetic retinopathy, choroidal neovascularization, or proliferative vitreoretinopathy is treated thereby.
